(19) United States
(12) Patent Application Publication
(10) Pub. No.: US 2026/0272857 A1
(43) Pub. Date: Sep. 17, 2026

(54) AN ORALLY RAPIDLY DISINTEGRATING TABLET OF PROPRANOLOL AND ITS PROCESS OF PREPARATION

(71) Applicant: NOVUMGEN LIMITED, London (GB)

(72) Inventor: Kamleshkumar Patel, London (GB)

(21) Appl. No.: 19/168,965

(22) PCT Filed: Mar. 27, 2024
(86) PCT No.: PCT/GB2024/050819

§ 371 Date: Sep. 25, 2025

(30) Foreign Application Priority Data
Mar. 29, 2023 (GB) 2304558.6

(51) Int. Cl. A61K 31/138; A61J 1/03; A61J 1/14; A61K 9/00

(52) U.S. Cl. CPC A61K 31/138; A61J 1/035; A61J 1/1468; A61K 9/0056

(57) ABSTRACT

The present invention relates to an Orally rapidly disintegrating tablet comprising Propranolol or pharmaceutically acceptable salts thereof, at least one disintegrant, at least one diluent, a lubricant, a sweetener, and a flavoring agent. The present invention is based on enhancing disintegration and dissolution, which is beneficial in the treatment of hypertension, angina, atrial fibrillation, myocardial infarction, migraine, essential tremor, hypertrophic subaortic stenosis, pheochromocytoma, proliferating infantile hemangioma and very convenient for administration for solid dosage form without swallowing difficulties, thereby increasing patient compliance.

### FIELD OF THE INVENTION

The present invention relates to a pharmaceutical composition of Propranolol. The present invention relates to an Orally rapidly disintegrating tablet of Propranolol or pharmaceutically acceptable salts thereof for oral administration. The present invention also relates to the process of the preparation of the same.

### BACKGROUND OF THE INVENTION

Propranolol was first disclosed in the U.S. Pat. No. 3,337,628. Propranolol is a nonselective, competitive antagonist at beta-adrenoceptors. It binds with high affinity to both beta-1 and beta-2 receptor subtypes but has lower affinity at the beta-3 subtype. Propranolol exerts its effects primarily by blocking the action of the endogenous catecholamines, epinephrine, and norepinephrine, at beta-adrenoceptors. Propranolol also blocks the effects of drugs with beta-adrenoceptor agonist activity.

Propranolol decreases heart rate, the force of contraction, and cardiac output. It prolongs systole by retarding conduction so that the synergy of contraction of ventricular fibers is disturbed. Cardiac work and oxygen consumption are reduced as the product of heart rate and aortic pressure decreases. Total coronary flow is reduced (blockade of dilator β receptors), but this is largely restricted to the subepicardial region, while perfusion of the subendocardial area is not affected.

Propranolol is used in the management and treatment of hypertension, to lower blood pressure which will reduce the risk of fatal and nonfatal cardiovascular events, primarily strokes and myocardial infarctions. It is also used to treat angina, atrial fibrillation, migraine, essential tremor, hypertrophic subaortic stenosis, pheochromocytoma, and proliferating infantile haemangioma.

The IUPAC name of Propranolol is 1-naphthalen-1-yloxy-3-(propan-2-ylamino) propan-2-ol. Propranolol hydrochloride is a high permeability and high solubility drug which makes it belong to BCS class I in the Biopharmaceutical classification system. The elimination half-life of propranolol is 8 hours. The plasma half-life of propranolol is 3 to 6 hours.

In US, Propranolol is available as tablets, capsules, oral concentrate, solutions, suspensions, and injectable forms. The marketed tablets of Propranolol are available in six strengths: 10 mg, 20 mg, 40 mg, 60 mg, 80 mg, and 90 mg. The extended-release capsules are available in four strengths: 60 mg, 80 mg, 120 mg, and 160 mg. The oral concentrate is available in 80 mg/ml. The parenteral dosage form of propranolol is available in 1 mg/ml. Liquid dosage forms of propranolol are available in four strengths: 4.28 mg/ml, 10 mg/ml 20 mg/ml, and 40 mg/ml. In UK, the oral solution is available in four strengths: 5 mg/5 ml, 10 mg/5 ml, 40 mg/5 ml, and 50 mg/5 ml.

U.S. Pat. No. 6,500,454 B1 discloses an oral capsule filled with sustained-release beads. The process of this invention involves preparing a propranolol-containing core coated with ethyl cellulose to form an ethyl cellulose first membrane around said core. The previously coated ethyl cellulose core is now coated with a mixture of plasticized ethyl cellulose and an enteric polymer. The Second membrane provides a lag time before drug release to form a Timed, Sustained Release coated drug particle, and then Time sustained release beads were filled in capsules.

U.S. Pat. No. 7,329,416 B2 discloses an oral sublingual tablet of propranolol. The process involves mixing propranolol powder with magnesium stearate. To the prepared mixture other components such as disodium hydrogen phosphate, Lactose, HPMC, and Sodium dihydrogen phosphate as buffering agents were added and then combined in a blender for blending. Further the blended composition is compressed into tablets.

U.S. Pat. No. 4,898,737A discloses the pellets to be filled into capsules or compressed into tablets which comprises a core containing a propranolol or a pharmaceutically acceptable salt thereof surrounded by polymeric material in an outer multi-layer membrane. The outer multilayer membrane is formed by film forming water insoluble and minor proportion of water soluble polymer in the ratio to provide release of propranolol from the pellet at a rate allowing controlled absorption thereof over a twenty-four-hour period.

U.S. Pat. No. 4,428,926 discloses an oral sustained-release tablet that comprises a core containing propranolol and a coating of polyvinyl alcohol and polyethylene glycol as the expandable lattice. The tablet containing propranolol consists of HPMC, Sugar, and Magnesium stearate further this tablet is coated with polyvinyl alcohol and polyethylene glycol which was previously mixed in methanol-water at 1:1 v/v.

EP3384899A1 discloses an oral aqueous non-alcoholic solution comprising propranolol, a non-sugar type sweetener, and is substantially free of an aromatic preservative agent. The composition contains saccharin sodium, the flavoring agent is a mixture of vanilla and strawberry, the viscosity-increasing agent is hydroxyethyl cellulose, and sodium propionate. The process of this invention involves adding and dissolving the above components into the purified water under stirring. After that, the pH of the solution was then adjusted with an aqueous solution of monohydrated citric acid. Finally, the bulk was brought to the final volume with the remaining amount of water and well mixed. Once filtered, the prepared solutions can be filled into amber glass bottles and closed with a child-resistant cap.

The Orally rapidly disintegrating tablet of the present invention is a pharmaceutical formulation that rapidly disintegrates in the mouth. The Orally rapidly disintegrating tablet of Propranolol is best suited dosage form to formulate, as it provides advantages like ease of administration of said solid dosage form in patients without ingestion of water, for patients who have difficulty in swallowing, such as the elderly, and dysphasic patients. In contrast, the liquid dosage form is bulky, difficult to transport, and takes up a lot of space. Due to their inherent instability, liquid dosage forms often have shorter shelf lives.

### SUMMARY OF THE INVENTION

In accordance with the present invention, the orally rapidly disintegrating tablet for oral administration is prepared. The orally rapidly disintegrating tablet comprises Propranolol or pharmaceutically acceptable salts thereof, at least one disintegrant, at least one diluent, and one or more pharmaceutically acceptable excipients.

Another embodiment relates to a pharmaceutical Orally rapidly disintegrating tablet which consists essentially of about 0.025% w/w to about 10% w/w, preferably in the range from about 0.05% w/w to about 6% w/w sodium starch glycolate, wherein the tablet exhibits disintegration within less than 3 minutes, preferably less than 2 minutes.

Another embodiment of the present invention is to provide a process for obtaining said Orally rapidly disintegrating tablet by direct compression method where the direct compression method is a process that involves sieving, mixing, and compression operations.

Further another embodiment of the present invention is to provide a method of preparing composition comprising: (a) Sieving propranolol hydrochloride or pharmaceutically acceptable salts thereof, a diluent and a disintegrant through 40 #sieve and sweetener, flavoring agent, and lubricant separately through 60 #sieve; (b) Blending diluent and propranolol hydrochloride in blender; (c) Mixing of Previously sifted disintegrant, sweetener, and flavoring agent, to the above blend; (d) Adding previously sifted lubricant to the above mixture of step (c) and blend in a blender; and (e) Compressing the lubricated mixture into the tablet dosage form.

Another embodiment of the present invention which effectively treat hypertension, angina, atrial fibrillation, myocardial infarction, migraine, essential tremor, hypertrophic subaortic stenosis, pheochromocytoma, and proliferating infantile hemangioma.

### OBJECTS OF THE INVENTION

The primary object of the present invention is to provide an Orally rapidly disintegrating tablet of Propranolol or pharmaceutically acceptable salts thereof.

Another object of the present invention is to provide a therapeutically effective amount of Propranolol or pharmaceutically acceptable salts thereof, sodium starch glycolate, mannitol, microcrystalline cellulose, magnesium stearate, and one or more pharmaceutically acceptable excipients.

A further object of the present invention is to enhance the disintegration and dissolution of the drug for oral administration.

A further object of the present invention is to provide a stable Orally rapidly disintegrating tablet of Propranolol or pharmaceutically acceptable salts thereof.

Yet another object of the present invention is to provide a treatment that is effective and very convenient for administration for elderly and dysphagic patients without the need for water and for people with swallowing difficulties, which will improve patient compliance.

### DETAILED DESCRIPTION OF THE INVENTION

The present invention is understood more readily by reading the following detailed description of the invention and by studying the included examples.

The term “orally rapidly disintegrating tablet” refers to a solid dosage form of the present invention, which disintegrates rapidly in the oral cavity of a patient after administration, without chewing. The rate of disintegration can vary but it is faster when compared to the conventional Solid dosage forms which are intended to be swallowed immediately after administration.

The main embodiment of the present invention is an Orally rapidly disintegrating tablet comprising of Propranolol or pharmaceutically acceptable salts thereof, at least one disintegrant, and at least one diluent. In addition, the orally rapidly disintegrating tablet further comprises at least one pharmaceutically acceptable excipient selected from the sweeteners, flavouring agents, and lubricants.

The term “about”, as and when used in this specification, means ±10% of the mentioned value.

As per one embodiment of the present invention Propranolol or pharmaceutically acceptable salts thereof, is present in the range of about 2% w/w to about 20% w/w, preferably in the range of about 5% w/w to about 15% w/w.

As per one embodiment, a suitable diluent for the present invention is selected from the group consisting of dextrates, microcrystalline cellulose, dextrose, fructose, Sorbitol, pregelatinized starch, starch, xylitol, sucrose, maltodextrin, maltose, mannitol or combinations thereof. In the present invention, microcrystalline cellulose is preferred as a diluent in the range from about 5% w/w to about 40% w/w, preferably from about 10% w/w to about 30% w/w, and mannitol is present in the range from about 40% w/w to about 80% w/w, preferably from about 50% w/w to about 70% w/w. In the present invention, the combination of microcrystalline cellulose and mannitol is preferred as a diluent in the range of about 55% w/w to about 95% w/w, preferably in the range from about 75% w/w to about 85% w/w. Mannitol as a filler provide its physicochemical properties, such as non-hygroscopicity, chemical inertness, and advantageous tableting behavior, including compactability. Mannitol has a mild, sweet pleasant taste with a cooling effect in the mouth. Microcrystalline cellulose promotes the efficient dry blending of ingredients and provides excellent compression of tablets. Therefore, the combination of microcrystalline cellulose and mannitol is preferred as diluents in comparison with other diluents in the present invention.

As per one another embodiment, a suitable disintegrant for the present invention is selected from the group consisting of methylcellulose, alginic acid, carboxymethylcellulose calcium, guar gum, carboxymethylcellulose Sodium, polacrilin potassium, croscarmellose sodium, poloxamer, sodium starch glycolate, and sodium alginate or combinations thereof. Sodium Starch glycolate is the preferred disintegrant for the present invention in the range from about 0.025% w/w to about 10% w/w, preferably in the range from about 0.05% w/w to about 6% w/w. In the present invention, the ratio of Sodium Starch glycolate to a combination of microcrystalline cellulose and mannitol is in the range of 1:10 to 1:70 and preferably it is in the range of 1:20 to 1:50. Sodium starch glycolate has good hydration capacity and flow properties. Further, it presents the tendency to absorb water rapidly, so it swells in a significant amount. Therefore, this rapid water absorption by sodium starch glycolate molecules has significant increase in the volume of granules resulting in rapid and uniform disintegration. Therefore, Sodium Starch glycolate is the disintegrant of choice for the Orally rapidly disintegrating tablet of Propranolol Hydrochloride.

As per one more embodiment of the present invention, a suitable lubricant is selected from the group consisting of boric acid, magnesium stearate, Sodium Stearyl fumarate, micronized polyoxy ethylene glycol, leucine, sodium benzoate, sodium acetate, sodium lauryl sulfate, stearic acid, sodium stearate, sodium oleate, calcium stearate, waxes or any combination thereof. Magnesium stearate is preferred as a lubricant for the present invention present in the range from about 0.05% w/w to about 10% w/w, preferably in the range from about 0.25% w/w to about 5% w/w, wherein the ratio of magnesium stearate to Propranolol hydrochloride is 1:1 to 1:15 preferably in the range of about 1:3 to 1:10.

As per another embodiment of the present invention, at least one further pharmaceutically acceptable excipient is a sweetener selected from the group consisting of cyclamate, acesulfame potassium, neo hesperidin dihydrochalcone, monoammonium glycyrrhizinate, saccharin sodium, sucralose, saccharin, aspartame or any combination thereof. Aspartame is preferred as a sweetener for the present invention in the range of about 0.25% w/w to about 10% w/w, preferably in the range of about 0.5% w/w to about 5% w/w.

As per one another embodiment of the present invention, a flavoring agent is selected from the group consisting of menthol, floral fennel flavor, mint powder, vanillin, or orange flavor or combinations thereof. The orange flavor is preferred as a flavoring agent present in the range from about 0.05% w/w to about 10% w/w, preferably in the range from about 0.5% w/w to about 5% w/w.

As per the preferred embodiment, the orally rapidly disintegrating tablet of propranolol is used for the treatment or prevention of hypertension, angina, atrial fibrillation, myocardial infarction, migraine, essential tremor, hypertrophic subaortic stenosis, pheochromocytoma, proliferating infantile haemangioma.

As per one embodiment of the present invention, the orally rapidly disintegrating tablet comprising Propranolol or pharmaceutically acceptable salts thereof, present in an amount from about 2% w/w to about 20% w/w, preferably in the range of about 5% w/w to about 15% w/w, a diluent is present in the range of about 55% w/w to about 95% w/w, preferably in the range from about 75% w/w to about 85% w/w, a disintegrant is present in the range from about 0.025% w/w to about 10% w/w, preferably in the range from about 0.05% w/w to about 6% w/w, lubricant is present in the range from about 0.05% w/w to about 10% w/w, preferably in the range from about 0.25% w/w to about 5% w/w, the sweetener is present in the range of about 0.25% w/w to about 10% w/w, preferably in the range of about 0.5% w/w to about 5% w/w and the flavoring agent is present in the range from about 0.05% w/w to about 10% w/w, preferably in the range from about 0.5% w/w to about 5% w/w.

As per the preferred embodiment of the present invention, the orally rapidly disintegrating tablet comprising Propranolol or pharmaceutically acceptable salts thereof, present in an amount from about 2% w/w to about 20% w/w, preferably in the range of about 5% w/w to about 15% w/w, a combination of microcrystalline cellulose and mannitol present in the range of about 55% w/w to about 95% w/w, preferably in the range of about 75% w/w to about 85% w/w, sodium starch glycolate is present in the range from about 0.025% w/w to about 10% w/w, preferably in the range of about 0.05% w/w to about 6% w/w, magnesium stearate is present in the range from about 0.05% w/w to about 10% w/w, preferably in the range from about 0.25% w/w to about 5% w/w, aspartame is present in the range of about 0.25% w/w to about 10% w/w, preferably in the range of about 0.5% w/w to about 5% w/w, and the orange flavor is present in the range from about 0.05% w/w to about 10% w/w, preferably in the range from about 0.5% w/w to about 5% w/w.

As per one more embodiment of the present invention, the ratio of disintegrant to diluent is in the range of 1:10 to 1:70 and preferably it is in the range of 1:20 to 1:50. The ratio of sodium starch glycolate to a combination of microcrystalline cellulose and mannitol is in the range of 1:10 to 1:70 and preferably it is in the range of 1:20 to 1:50. The ratio of lubricant to the drug is in the range of 1:1 to 1:5, preferably it is in the range of about 1:3 to 1:10. The ratio of magnesium stearate to propranolol hydrochloride is in the range of 1:1 to 1:15, preferably it is in the range of about 1:3 to 1:10.

Another embodiment of the present invention is to manufacture an Orally rapidly disintegrating tablet containing Propranolol by direct compression method, which is one of the most economical methods.

As per one another embodiment of the present invention, the disintegrating time of the Propranolol Orally rapidly disintegrating tablet is less than 3 minutes, preferably less than 2 minutes.

As per another embodiment of the present invention, 90% of the Propranolol is released in 30 minutes, preferably more than 90% is released within 15 minutes.

As per one embodiment of the present invention, a suitable packaging material for an Orally rapidly disintegrating tablet of Propranolol is selected from the group consisting of a Polypropylene Bottle with Silica canister, PP Bottle with an Oxygen scavenger, HDPE Bottle, HDPE Bottle with Child Resistant cap, HDPE Bottle with Silica canister, HDPE Bottle with an Oxygen scavenger, Alu-Alu Blister, and PVC/PVDC-Alu Film. In the present invention, the PVC/PVDC-Alu Film or HDPE Bottle with Child Resistant cap is preferred as packaging material to deliver the desired physicochemical parameters.

As per one more embodiment, the direct compression method is used to manufacture the orally rapidly disintegrating tablet of Propranolol. Propranolol Hydrochloride, Mannitol, Microcrystalline cellulose, and Sodium starch glycolate are sieved separately through 40 #sieve. Aspartame, orange flavor, and magnesium stearate are sieved separately through 60 #sieve. In blender, mannitol, microcrystalline cellulose, and Propranolol Hydrochloride are blended. After that, previously sifted Aspartame, Sodium starch glycolate, and orange flavor are added to the above blend. At last, the previously sifted magnesium stearate is added to the above mixture and blended. Finally, the lubricated mixture is compressed to form tablets. The prepared tablets were packed in PVC/PVDC-Alu Film or HDPE Bottle with Child Resistant cap preferred as packaging materials to deliver the desired physicochemical parameters.

The invention is further illustrated by the following examples, which are by no means intended to limit the scope of the invention but are given by way of illustration.

### Example 1

The Orally rapidly disintegrating tablet was manufactured according to the method defined below with ingredients shown in table I for different dose strengths are 10 mg, 20 mg, 40 mg, 60 mg, 80 mg, and 90 mg of Propranolol:

TABLE I

| Ingredients | % w/w |
|---|---|
| Propranolol hydrochloride | 11.11 |
| Mannitol | 52.17 |
| Microcrystalline Cellulose | 27.78 |
| Sodium starch glycolate | 2.00 |
| Aspartame | 2.22 |
| Orange flavor | 1.39 |
| Magnesium Stearate | 3.33 |
| Total | 100.00 |

### Manufacturing Process:

Propranolol Hydrochloride, Mannitol, Microcrystalline cellulose, and Sodium starch glycolate are sieved separately through 40 #sieve. Aspartame, orange flavor, and magnesium stearate are sieved separately through 60 #sieve. In blender, mannitol, microcrystalline cellulose, and Propranolol Hydrochloride are blended. After that, previously sifted Aspartame, Sodium starch glycolate, and the orange flavor are added to the above blend. At last, the previously sifted magnesium stearate is added to the above mixture and blended. The blend flow was not good so, further compression activity was not performed.

To optimize blend flow, it was necessary to change the grade of microcrystalline cellulose.

| Test Parameters | |
|---|---|
| of blend | Result |
| Bulk Density (gm/ml) | 0.532 |
| Tap Density (gm/ml) | 0.806 |
| Carr Index (%) | 34.04 |
| Hausner Ratio | 1.52 |

### Example 2

The Orally rapidly disintegrating tablet was manufactured according to the method defined below with ingredients shown in table II for different dose strengths are 10 mg, 20 mg, 40 mg, 60 mg, 80 mg, and 90 mg of Propranolol:

TABLE II

| Ingredients | % w/w |
|---|---|
| Propranolol hydrochloride | 11.11 |
| Mannitol | 52.17 |
| Microcrystalline Cellulose | 27.78 |
| Sodium starch glycolate | 2.00 |
| Aspartame | 2.22 |
| Orange flavor | 1.39 |
| Magnesium Stearate | 3.33 |
| Total | 100.00 |

### Manufacturing Process:

Propranolol Hydrochloride, Mannitol, Microcrystalline cellulose, and Sodium starch glycolate are sieved separately through 40 #sieve. Aspartame, orange flavor, and magnesium stearate are sieved separately through 60 #sieve. In blender, mannitol, microcrystalline cellulose and Propranolol Hydrochloride are blended. After that, previously sifted Aspartame, Sodium starch glycolate, and the orange flavor are added to the above blend and mixed. At last, the previously sifted magnesium stearate is added to the above mixture and blended. Finally, the lubricated mixture is compressed to form tablets.

Weight variation was observed during compression due to blend flow issues.

To optimize blend flow, it was necessary to increase the concentration of mannitol.

| Test Parameters | |
|---|---|
| of blend | Result |
| Bulk Density (gm/ml) | 0.588 |
| Tap Density (gm/ml) | 0.811 |
| Carr Index (%) | 27.45 |
| Hausner Ratio | 1.38 |

The tablets present the characteristics mentioned in the table below:

| Test Parameters | Specification | Result |
|---|---|---|
| Hardness | 30N to 80N | 38N |
| Disintegration | NMT 3 min | 38 sec |

### Example 3

The Orally rapidly disintegrating tablet was manufactured according to the method defined below with ingredients shown in table III for different dose strengths are 10 mg, 20 mg, 40 mg, 60 mg, 80 mg, and 90 mg of Propranolol:

TABLE III

| Ingredients | % w/w |
|---|---|
| Propranolol hydrochloride | 11.11 |
| Mannitol | 62.03 |
| Microcrystalline Cellulose | 17.91 |
| Sodium starch glycolate | 2.00 |
| Aspartame | 2.22 |
| Orange flavor | 1.39 |
| Magnesium Stearate | 3.33 |
| Total | 100.00 |

### Manufacturing Process:

Propranolol Hydrochloride, Mannitol, Microcrystalline cellulose, and Sodium starch glycolate are sieved separately through 40 #sieve. Aspartame, orange flavor, and magnesium stearate are Sieved separately through 60 #sieve. In blender, mannitol, microcrystalline cellulose, and Propranolol Hydrochloride are blended. After that, previously sifted Aspartame, Sodium starch glycolate, and the orange flavor are added to the above blend. At last, the previously sifted magnesium stearate is added to the above mixture and blended. Finally, the lubricated mixture is compressed to form tablets.

The blend flow was proper but a Capping problem was observed during compression.

To minimize the Capping problem, it was necessary to decrease the concentration of Lubricant.

| Test Parameters | |
|---|---|
| of blend | Result |
| Bulk Density (gm/ml) | 0.521 |
| Tap Density (gm/ml) | 0.676 |
| Carr Index (%) | 22.92 |
| Hausner Ratio | 1.30 |

The tablets present the characteristics mentioned in the table below:

| Test Parameters | Specification | Result |
|---|---|---|
| Hardness | 30N to 80N | 47N |
| Disintegration | NMT 3 min | 29 sec |

### Example 4

The Orally rapidly disintegrating tablet was manufactured according to the method defined below with ingredients shown in table IV for different dose strengths are 10 mg, 20 mg, 40 mg, 60 mg, 80 mg, and 90 mg of Propranolol:

TABLE IV

| Ingredients | % w/w |
|---|---|
| Propranolol hydrochloride | 11.11 |
| Mannitol | 63.70 |
| Microcrystalline Cellulose | 17.91 |
| Sodium starch glycolate | 2.00 |
| Aspartame | 2.22 |
| Orange flavor | 1.39 |
| Magnesium Stearate | 1.67 |
| Total | 100.00 |

### Manufacturing Process:

Propranolol Hydrochloride, Mannitol, Microcrystalline cellulose and Sodium starch glycolate are sieved separately through 40 #sieve. Aspartame, orange flavor, and magnesium stearate are Sieved separately through 60 #sieve. In blender, mannitol, microcrystalline cellulose, and Propranolol Hydrochloride are blended. After that, previously sifted Aspartame, Sodium starch glycolate, and the orange flavor are added to the above blend and mixed. At last, the previously sifted magnesium stearate is added to the above mixture and blended. Finally, the lubricated mixture is compressed to form tablets. The prepared tablets were packed in PVC/PVDC-Alu Film or HDPE Bottle with Child Resistant cap preferred as packaging materials to deliver the desired physicochemical parameters.

All the physical and chemical parameters of the tablets were found satisfactory.

| Test Parameters | |
|---|---|
| of blend | Result |
| Bulk Density (gm/ml) | 0.513 |
| Tap Density (gm/ml) | 0.667 |
| Carr Index (%) | 23.08 |
| Hausner Ratio | 1.30 |

### Example 5

### The Dissolution Profile of the Tablet Prepared According to Example 4

The conditions of dissolution are the following:
Apparatus: USP type II (paddle)
Rate of rotation: 50 RPM
Volume: 500 ml
Temperature: 37° C.±0.5° C.
Detection: High-performance liquid chromatography equipped with UV/PDA at 292 nm
Dissolution medium: 0.1N Hydrochloric Acid

The orally rapidly disintegrating tablet of propranolol was tested for its dissolution profile measured in 500 mL of 0.1 N HCl at 50 RPM in USP II (Paddle) apparatus and the active ingredient of the tablet is released in more than 90% in 15 minutes.

### Example 6

The tablets prepared according to example 4 were subjected to a stability study of 25° C./60% RH and 40° C./75% RH for 3 and 6 months. Results are tabulated below.

### Propranolol 10 mg Orally Rapidly Disintegrating Tablet

| | | | Exp 4 (After | | Exp 4 (After | |
|---|---|---|---|---|---|---|
| | | | 3 months) | | 6 months) | |
| | | Exp 4 | 25° C./ | 40° C./ | 25° C./ | 40° C./ |
| Test | Specification | (Initial) | 60% RH | 75% RH | 60% RH | 75% RH |
| Hardness | 20N-60N | 38N | 33N | 36N | 42N | 35N |
| Disintegration | NMT 3 min | 55 sec | 51 sec | 48 sec | 51 sec | 55 sec |
| Assay (%) | 95.0%-105.0% | 99.3 | 99.6 | 98.8 | 99.1 | 98.9 |
| Dissolution | NLT 75% (Q) | 95.4 | 98.4 | 98.8 | 97.8 | 98.1 |
| | labeled | | | | | |
| | amount of | | | | | |
| | Propranolol | | | | | |
| | Hydrochloride | | | | | |
| | should be | | | | | |
| | dissolved in | | | | | |
| | 15 minutes. | | | | | |

### Propranolol 40 mg Orally Rapidly Disintegrating Tablet

| | | | Exp 4 (After | | Exp 4 (After | |
|---|---|---|---|---|---|---|
| | | | 3 months) | | 6 months) | |
| | | Exp 4 | 25° C./ | 40° C./ | 25° C./ | 40° C./ |
| Test | Specification | (Initial) | 60% RH | 75% RH | 60% RH | 75% RH |
| Hardness | 30N to 80N | 45N | 41N | 49N | 53N | 48N |
| Disintegration | NMT 3 min | 23 sec | 29 sec | 25 sec | 34 sec | 22 sec |
| Assay (%) | 95.0%-105.0% | 101.4 | 98.9 | 97.8 | 98.1 | 101.6 |
| Dissolution | NLT 75% (Q) | 97.6 | 90.4 | 92.6 | 93.4 | 90.2 |
| | labeled | | | | | |
| | amount of | | | | | |
| | Propranolol | | | | | |
| | Hydrochloride | | | | | |
| | should be | | | | | |
| | dissolved in | | | | | |
| | 15 minutes. | | | | | |

1. An Orally rapidly disintegrating tablet of propranolol for oral administration comprising:
a) propranolol or pharmaceutically acceptable salts thereof, is present in an amount ranging from about 2% w/w to about 20% w/w, preferably in the range from about 5% w/w to about 15% w/w;
b) at least one diluent;
c) at least one disintegrant is present in the range of about 0.025% w/w to about 10% w/w, preferably in the range of about 0.05% w/w to about 6% w/w;
d) at least one lubricant is present in the range from about 0.05% w/w to about 10% w/w, preferably in the range from about 0.25% w/w to about 5% w/w; and
e) at least one or more pharmaceutically acceptable excipients.

2. The Orally rapidly disintegrating tablet according to claim 1, wherein the diluent is selected from the group consisting of dextrates, microcrystalline cellulose, dextrose, fructose, Sorbitol, pregelatinized starch, starch, xylitol, sucrose, maltodextrin, maltose, mannitol or combinations thereof.

3. The Orally rapidly disintegrating tablet according to claim 2, wherein the diluent is mannitol present in the range from about 40% w/w to about 80% w/w, preferably from about 50% w/w to about 70% w/w.

4. The Orally rapidly disintegrating tablet according to claim 2, wherein the diluent is microcrystalline cellulose present in the range from about 5% w/w to about 40% w/w, preferably from about 10% w/w to about 30% w/w.

5. The Orally rapidly disintegrating tablet according to claim 2, wherein the diluent is a combination of microcrystalline cellulose and mannitol present in the range of about 55% w/w to about 95% w/w, preferably in the range of about 75% w/w to about 85% w/w.

6. The Orally rapidly disintegrating tablet according to claim 1, wherein the disintegrant is selected from the group consisting of methylcellulose, alginic acid, carboxymethylcellulose calcium, carboxymethylcellulose sodium, polacrilin potassium, croscarmellose sodium, guar gum, poloxamer, sodium starch glycolate, and sodium alginate or any combination thereof.

7. The Orally rapidly disintegrating tablet according to claim 6, wherein the disintegrant is Sodium starch glycolate.

8. The Orally rapidly disintegrating tablet according to claim 1, wherein the ratio of a disintegrant to diluent is in the range of 1:10 to 1:70 and preferably it is in the range of 1:20 to 1:50.

9. The Orally rapidly disintegrating tablet according to claim 8, wherein the ratio of Sodium starch glycolate to a combination of microcrystalline cellulose and mannitol is in the range of 1:10 to 1:70 and preferably in the range of 1:20 to 1:50.

10. The Orally rapidly disintegrating tablet according to claim 1, wherein the lubricant is selected from the group consisting of boric acid, magnesium stearate, sodium Stearyl fumarate, micronized polyoxyethylene glycol, leucine, sodium benzoate, sodium acetate, sodium lauryl sulfate, stearic acid, sodium stearate, sodium oleate, calcium stearate or combinations thereof.

11. The Orally rapidly disintegrating tablet according to claim 10, wherein the lubricant is magnesium stearate.

12. The Orally rapidly disintegrating tablet according to claim 1, wherein the ratio of a lubricant to the drug is in the range of 1:1 to 1:15, preferably it is in the range of about 1:3 to 1:10.

13. The Orally rapidly disintegrating tablet according to claim 12, wherein the ratio of magnesium stearate to propranolol hydrochloride is in the range of 1:1 to 1:15 and preferably in the range of 1:3 to 1:10.

14. The Orally rapidly disintegrating tablet according to claim 1, wherein one or more pharmaceutically acceptable excipients, are selected from the sweeteners or flavoring agents.

15. The Orally rapidly disintegrating tablet according to claim 14, wherein the sweetener is selected from the group consisting of cyclamate, acesulfame potassium, neo hesperidin dihydrochalcone, monoammonium glycyrrhizinate, saccharin sodium, sucralose, saccharin, aspartame or combinations thereof is present in the range of about 0.25% w/w to about 10% w/w, preferably in the range of about 0.5% w/w to about 5% w/w.

16. The Orally rapidly disintegrating tablet according to claim 14, wherein the flavoring agent is selected from the group consisting of menthol, floral fennel flavor, mint powder, vanillin, or orange flavor or combinations thereof is present in the range from about 0.05% w/w to about 10% w/w, preferably in the range from about 0.5% w/w to about 5% w/w.

17. The Orally rapidly disintegrating tablet according to claim 14, further comprises aspartame and orange flavor.

18. The Orally rapidly disintegrating tablet according to claim 1, wherein the Orally rapidly disintegrating tablet is manufactured by the direct compression method comprising the steps of:
(a) Sieving propranolol hydrochloride or pharmaceutically acceptable salts thereof, Mannitol, Microcrystalline cellulose, and Sodium Starch glycolate separately through a 40 #sieve and Aspartame, orange flavor, and magnesium stearate separately through a 60 #sieve;
(b) Blending mannitol, microcrystalline cellulose, and propranolol hydrochloride in a blender;
(c) Mixing of Previously sifted sodium starch glycolate, aspartame, and orange flavor to the above blend;
(d) Adding previously sifted magnesium stearate to the above mixture of step (c) and blend in a blender;
(e) Compressing the lubricated mixture into the tablet dosage form; and
(f) Packing of the tablet into a PVC/PVDC-Alu Film or HDPE Bottle with Child Resistant cap.

19. The Orally rapidly disintegrating tablet according to claim 1, wherein the Orally rapidly disintegrating tablet is disintegrated upon contact with saliva in less than 3 minutes, preferably less than 2 minutes.

20. The Orally rapidly disintegrating tablet according to claim 1, wherein 90% of the propranolol or pharmaceutically acceptable salts thereof, is released within 30 minutes, preferably more than 90% is released within 15 minutes.

21. The Orally rapidly disintegrating tablet according to claim 1, is used for the treatment of hypertension, angina, atrial fibrillation, myocardial infarction, migraine, essential tremor, hypertrophic subaortic stenosis, pheochromocytoma, and proliferating infantile hemangioma.

22. The Orally rapidly disintegrating tablet according to claim 1, wherein the packaging material of the container is selected from the group consisting of Polypropylene Bottle with Silica canister, PP Bottle with an Oxygen scavenger, HDPE Bottle, HDPE Bottle with Child Resistant cap, HDPE Bottle with Silica canister, HDPE Bottle with Oxygen scavenger, Alu-Alu Blister, and PVC/PVDC-Alu Film.

23. The Orally rapidly disintegrating tablet according to claim 23, wherein the tablet is more stable for at least three months and six months at 25° C./60% relative humidity and at 40° C./75% relative humidity in PVC/PVDC-Alu Film or HDPE Bottle with Child Resistant cap.

24. The Orally rapidly disintegrating tablet according to claim 1, wherein:
a) 2% w/w to about 20% w/w, preferably in the range of about 5% w/w to about 15% w/w of propranolol or pharmaceutically acceptable salts thereof;
b) 5% w/w to about 40% w/w, preferably from about 10% w/w to about 30% w/w of microcrystalline cellulose;
c) 40% w/w to about 80% w/w, preferably from about 50% w/w to about 70% w/w of mannitol;
d) a combination of microcrystalline cellulose and mannitol present in the range of about 55% w/w to about 95% w/w, preferably in the range of about 75% w/w to about 85% w/w;
e) 0.025% w/w to about 10% w/w, preferably in the range of about 0.05% w/w to about 6% w/w of sodium starch glycolate;
f) 0.05% w/w to about 10% w/w, preferably in the range from about 0.25% w/w to about 5% w/w of magnesium stearate;
g) 0.25% w/w to about 10% w/w, preferably in the range of about 0.5% w/w to about 5% w/w of aspartame; and
h) 0.05% w/w to about 10% w/w, preferably in the range from about 0.5% w/w to about 5% w/w of orange flavor.
